(19) United States
(12) Patent Application Publication
(10) Pub. No.: US 2024/0273683 A1
(43) Pub. Date: Aug. 15, 2024

(54) CELL IMAGE ANALYSIS METHOD

(71) Applicant: SHIMADZU CORPORATION, Kyoto-Shi, Kyoto (JP)

(72) Inventors: Ryuji SAWADA, Kyoto-Shi, Kyoto (JP); Shuhei YAMAMOTO, Kyoto-Shi, Kyoto (JP)

(73) Assignee: SHIMADZU CORPORATION, Kyoto-Shi, Kyoto (JP)

(21) Appl. No.: 18/568,342

(22) PCT Filed: Jul. 28, 2022
(86) PCT No.: PCT/JP2022/029115

§ 371 Date: Dec. 8, 2023

(30) Foreign Application Priority Data
Jul. 29, 2021 (JP) 2021-124723

(51) Int. Cl. G06T 5/60; G06T 5/20; G06T 7/00

(52) U.S. Cl. CPC G06T 5/60; G06T 5/20; G06T 7/0012; G06T 2207/20032; G06T 2207/20081; G06T 2207/20084; G06T 2207/30024; G06T 2207/30204

(57) ABSTRACT

A cell image analysis method according to this invention includes a step of acquiring a cell image (10) including a cell (90); a step of generating a background component image (14) that extracts a distribution of a brightness component of a background (91) by filtering the cell image; a step of generating a corrected cell image (11) that is corrected based on the cell image and the background component image to reduce unevenness of brightness; and a first estimation step of estimating whether the cell in the image is a normal cell or an abnormal cell by using the corrected cell image and a learned model (6) that has learned to analyze the cell.

### TECHNICAL AREAS

The invention relates to a cell image analysis method, in particular to a cell analysis method for analyzing cells by using a learned model.

### BACKGROUND ART

Cell analysis methods for analyzing cells by using a learned model are known in the art. Such a cell analysis method is disclosed in International Publication No. WO 2019-171546, for example.

International Publication No. WO 2019-171546 discloses a cell image analysis method for analyzing images of cells captured by imaging an apparatus. Specifically, International Publication No. WO 2019-171546 discloses a configuration in which images of cells cultivated on a cultivation plate are captured by an imaging device such as a microscope to acquire cell images. The analysis method for analyzing cell images disclosed in International Publication No. WO 2019-171546 classifies the cells into normal and abnormal cells based on analysis results of the learned model. Also, International Publication No. WO 2019-171546 discloses a configuration in which each cell is classified into one of the normal cell and the abnormal cell by segmentation for classifying each of pixels of the cell into one of categories.

### PRIOR ART

### Patent Document

Patent Document 1: International Publication No. WO 2019-171546

### SUMMARY OF THE INVENTION

### Problems to be Solved by the Invention

Although not stated in International Publication No. WO 2019-171546, in a case in which cells are cultivated by using cultivation plates (cultivation containers), cultivation solution can make brightness of a background of a cell image uneven. A height of a liquid surface of the cultivation solution varies in a near-edge area of the cultivation container, specifically, the height of the liquid surface of the cultivation solution is increased toward the near-edge area of the cultivation container by surface tension. Such height variation of the liquid surface of the cultivation solution makes brightness of the background uneven. When a cell image that includes uneven brightness of a background is analyzed by using a learned model, accuracy of the analysis is reduced. For this reason, there is a need for a cell image analysis method that can prevent reduction of accuracy of analysis by a learned models even in a case in which brightness of a background of a cell image is uneven.

The present invention is intended to solve the above problem, and one object of the present invention is to provide a cell image analysis method capable of preventing reduction of accuracy of analysis by a learned models even in a case in which brightness of a background of a cell image is uneven.

### Means for Solving the Problems

In order to attain the aforementioned object, a cell image analysis method according to an aspect of the present invention includes a step of acquiring a cell image including a cell; a step of generating a background component image that extracts a distribution of a brightness component of a background from the cell image by filtering the acquired cell image; a step of generating a corrected cell image that is acquired by correcting the cell image based on the cell image and the background component image to reduce unevenness of brightness; and a first estimation step of estimating whether the cell in the image is a normal cell or an abnormal cell by using the corrected cell image and a learned model that has learned to analyze the cell.

### Effect of the Invention

As discussed above, the cell image analysis method according to the aforementioned aspect includes a step of generating a corrected cell image that is acquired by correcting the cell image to reduce unevenness of brightness; and a first estimation step of estimating whether the cell in the image is a normal cell or an abnormal cell by using the corrected cell image and a learned model that has learned to analyze the cell. Accordingly, this cell image analysis method can estimate whether a cell in an image is a normal cell or an abnormal cell by using the corrected cell image generated to reduce unevenness of brightness and the learned model. Consequently, it is possible to prevent reduction of accuracy of analysis by the learned model caused by unevenness of brightness of the background of the cell image.

### BRIEF DESCRIPTION OF THE DRAWINGS

FIG. 1 is a schematic diagram entirely showing a configuration of a cell image analysis apparatus according to an embodiment.

FIG. 2 is a schematic view illustrating a cell image.

FIG. 3 is a schematic view illustrating unevenness of brightness of the cell image caused by cultivation solution with which a cultivation container filled.

FIG. 4 is a schematic diagram illustrating a leaning method for learning a learning model, and a method for analyzing a cell image by using the learning model, which has learned to analyze cell images, according to the embodiment.

FIG. 5 is a schematic diagram illustrating a configuration in which a background component image is generated by an image processor according to the embodiment.

FIG. 6 is a schematic diagram illustrating a configuration in which a corrected cell image is generated by an image processor according to the embodiment.

FIG. 7 is a schematic diagram illustrating a configuration in which an image analyzer generates a first area estimation image by using a first learned model in the embodiment.

FIG. 8 is a schematic diagram illustrating a configuration in which an image processor generates a first superimposed cell image by using the corrected cell image and the first area estimation image in the embodiment.

FIG. 9 is a schematic view illustrating a superimposed cell image in a comparative example.

FIG. 10 is a schematic diagram illustrating a configuration in which the image processor generates a second area estimation image by using a second learned model in the embodiment.

FIG. 11 is a schematic diagram illustrating a configuration in which the image processor generates a second superimposed cell image by using the cell image and the second area estimation image in the embodiment.

FIG. 12 is a flowchart illustrating display processing of the superimposed cell image displayed by the cell image analysis apparatus according to the embodiment.

FIG. 13 is a flowchart illustrating generation processing of the background component image generated by the cell image analysis apparatus according to the embodiment.

### MODES FOR CARRYING OUT THE INVENTION

Embodiments embodying the present invention will be described with reference to the drawings.

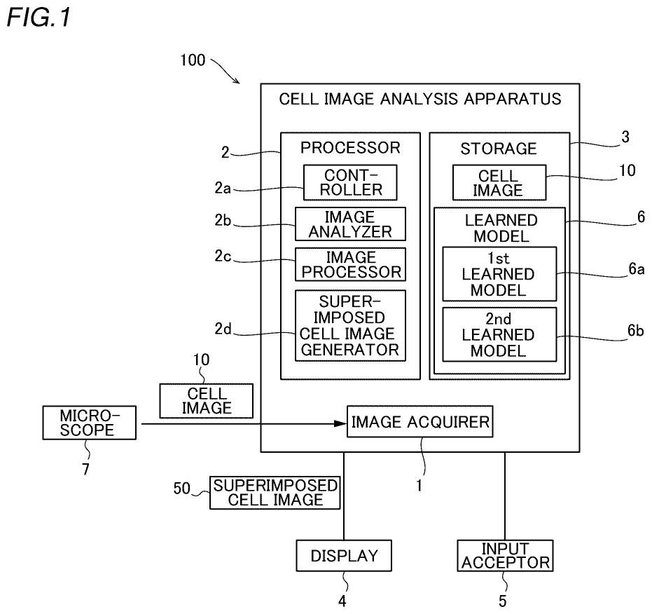

A configuration of a cell image analysis apparatus 100 according to an embodiment is now described with reference to FIG. 1.

### (Configuration of Cell Image Analysis Apparatus)

The cell image analysis apparatus 100 includes an image acquirer 1, a processor 2, a storage 3, a display 4, and an input acceptor 5 as shown in FIG. 1.

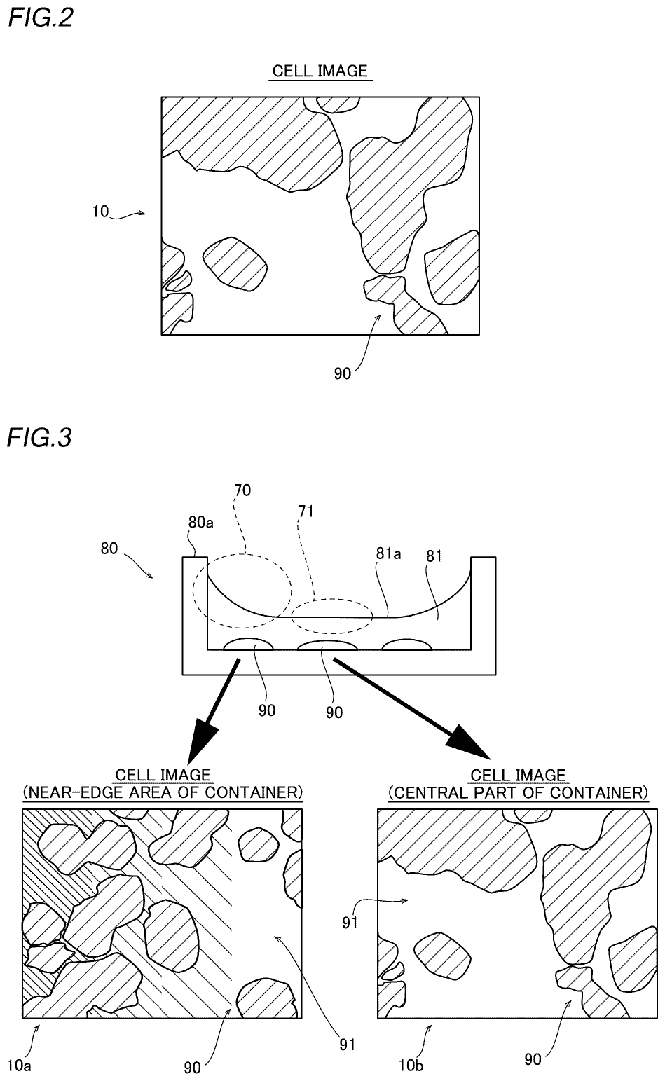

The image acquirer 1 is configured to acquire cell images 10. Each cell image 10 includes cells 90 (see FIG. 2). Specifically, the cell image 10 is an image including cultivated cells 90 cultivated in a cultivation container 80 (see FIG. 3) filled the cultivation solution 81 (see FIG. 3). In this embodiment, the Image acquirer 1 is configured to acquire the cell image 10 from a device that is configured to capture the cell image 10 such as a microscope 7 to which an imaging apparatus is attached, for example. The image acquirer 1 includes an input/output interface, for example.

The processor 2 is configured to analyze the acquired cell images 10. The processor 2 can include a CPU (Central Processing Unit), a ROM (Read Only Memory), a RAM (Random Access Memory), a graphics processing unit (GPU), a field-programmable gate array (FPGA) configured for image processing, etc. Also, the processor 2, which is hardware, etc., includes a constructed of a CPU as controller 2a, an image analyzer 2b, an image processor 2c and a superimposed cell image generator 2d as functional blocks of software (programs). The processor 2 can serve as the controller 2a, the image analyzer 2b, the image processor 2c, and the superimposed cell image generator 2d by executing programs stored in the storage 3. The controller 2a, the image analyzer 2b, the image processor 2c, and the superimposed cell image generator 2d may be individually constructed of a dedicated processor (processing circuit) as hardware.

The controller 2a is configured to control the cell image analysis apparatus 100. Also, the controller 2a is configured to direct the display 4 to display a superimposed cell image 50. The superimposed cell image 50 will be described later.

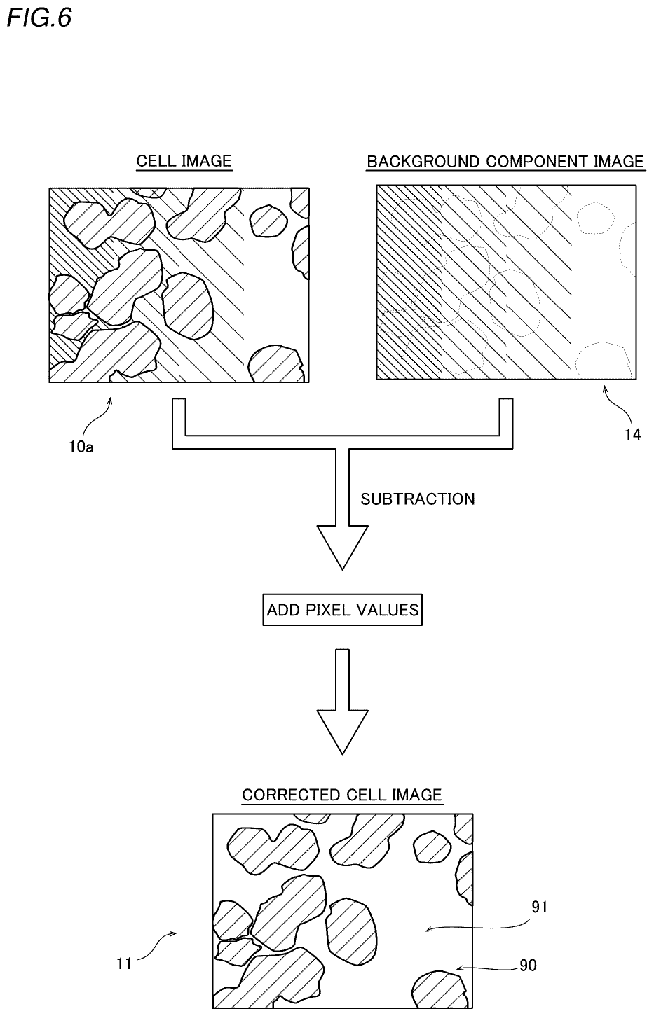

In this embodiment, the image analyzer 2b is configured to analyze whether each cell 90 included in the cell image 10 (see FIG. 2) is a normal cell or an abnormal cell. Specifically, the image analyzer 2b is configured to use a learned model 6 that has learned to analyze the cells 90 to analyze whether each cell 90 included in the cell image 10 is a normal cell or an abnormal cell. The learned model 6 also includes a first learned model 6a used to analyze a corrected cell image 11 (see FIG. 6), and a second learned model 6b is used to analyze the cell image 10. The normal and abnormal cells, the first learned model 6a, and the second learned model 6b will be described in detail later.

The image processor 2c is configured to generate a background component image 14 (see FIG. 5) that extracts a distribution of a brightness component of a background 91 (see FIG. 3) from the cell image 10. The image processor 2c is also configured to generate the corrected cell image 11 (see FIG. 6) by correcting the cell image 10 to reduce unevenness of brightness. Processes of the image processor 2c for generating the background component images 14 and the corrected cell images 11 will be described later.

The superimposed cell image generator 2d is configured to generate the superimposed cell image 50 that can discriminate between normal and abnormal cells. A configuration in which the superimposed cell image 50 is generated by the superimposed cell image generator 2d will be described in detail later.

The storage 3 is configured to store the cell images 10, the first learned model 6a, and the second learned model 6b. Also, the storage 3 is configured to store various programs to be executed by the processor 2. The storage 3 includes an HDD (Hard Disk Drive) or a storage such as SSD (Solid State Drive), for example.

The display 4 configured is to display the superimposed cell image 50 generated by the superimposed cell image generator 2d. The display 4 includes a display device such as an LCD monitor, for example.

The input acceptor 5 is configured to accept operating inputs from a user. The input acceptor 5 includes an input device such as a computer mouse, keyboard, etc., for example.

### (Cell Image)

The cell image 10 is described with reference to FIG. 2. Each cell image 10 includes cultivated cells 90. In this embodiment, the cell image 10 is a microscopic image captured by a microscope 7 to which an imaging apparatus attached. Each cell image includes cells 90 that can differentiate (cell potency) as the cultivated cells 90. For example, the cells 90 include IPS cells (Induced Pluripotent Stem cells) and ES cells (Embryonic Stem cells). The image analyzer 2b is configured to analyze whether each cell 90 included in the cell image 10 (see FIG. 3) is an undifferentiated cell or a deviated cell. Undifferentiated cells refer to cells that have cell potency. Deviated cells refer to cells that are already differentiated cell and do not have cell potency. In this embodiment, an undifferentiated cell is referred to as a normal cell. Also, a deviated cell is referred to as an abnormal cell.

### (Unevenness of Brightness of the Cell Image Caused by Cultivation Solution)

The following description describes a case in which the cultivation solution 81 makes brightness of the background 91 of the cell images 10 uneven with reference to FIG. 3. In this embodiment, the cell image 10 includes a cell image 10a that has uneven brightness of the background 91, and a cell image 10b that has even brightness of the background 91.

As shown in FIG. 3, the cells 90 are cultivated cells cultivated in the cultivation container 80 filled with the cultivation solution 81. A height of a surface 81a of the cultivation solution 81 gradually varies due to surface tension in a near-edge area 70 of the cultivation container 80. In an exemplary case shown in FIG. 3, the height of the liquid surface 81a of the cultivation solution 81 increases toward the edge 80a of the cultivation container 80. In this case, height difference of the cultivation solution 81 causes uneven brightness of the background 91 shown in the cell image 10a of FIG. 3. The cell image 10a includes the cultivated cells 90 that are located in the near-edge area 70 of the cultivation container 80. The uneven brightness of the background 91 of the cell image 10a shown in FIG. 3 is represented different hatching patterns. The cell image 10a shown in FIG. 3 represents exemplary unevenness of brightness in which brightness values (pixel values) of the background 91 decrease from the left side to the right side of the image. The near-edge area 70 of the cultivation container 80 includes a position of the edge 80a of the cultivation container 80 and an area near the position of the edge 80a of the cultivation container 80.

As shown in FIG. 3, the height of the liquid surface 81a of the cultivation solution 81 is constant in a central part 71 of the cultivation container 80. In a case in which the height of the liquid surface 81a of the cultivation solution 81 is constant, brightness of the background 91 does not become uneven. In other words, as shown in the cell image 10b, brightness of the background 91 is even in an image including the cultivated cells 90 that are located in the central part 71 of the cultivation container 80. The cell image 10b includes only the cultivated cells 90 that are located in the central part 71 of the cultivation container 80.

In a case in which the cell image 10a including uneven brightness of the background 91 is used for analysis of cells analyzed by using the learned model 6, the unevenness of brightness of the background 91 can reduce accuracy of the analysis. To address this, the image processor 2c generates the corrected cell image 11 generated to reduce unevenness of brightness of the background 91. The image analyzer 2b is configured to analyze the cells by using the corrected cell image 11 generated by the image processor 2c, and the learned model 6.

### (Image Analysis Method)

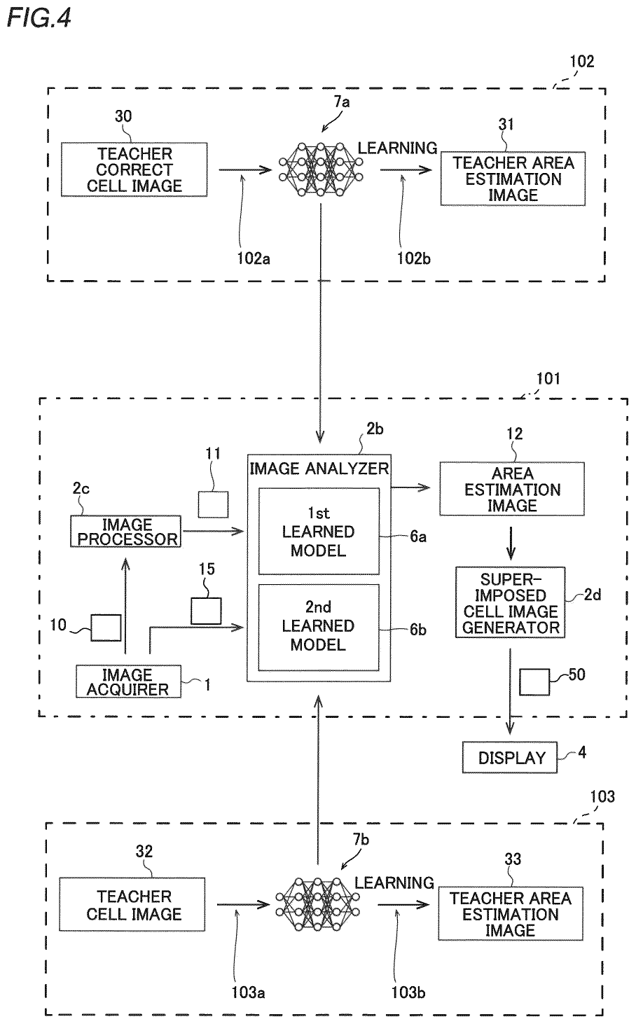

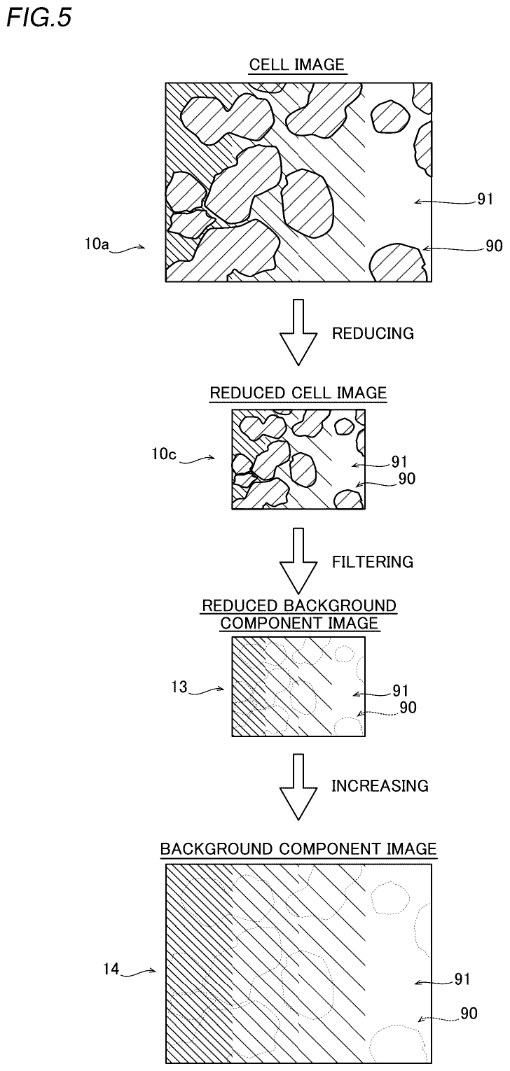

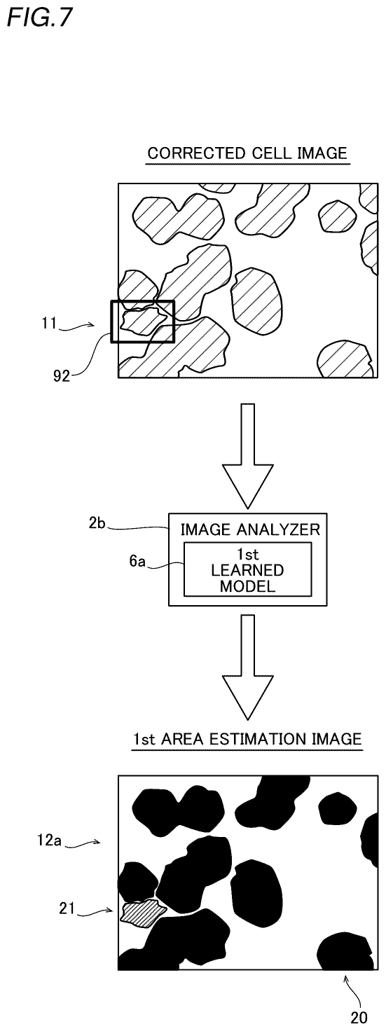

A method for analyzing the cell images 10 by using the cell image analysis method according to this embodiment is now described with reference to FIG. 4. A configuration in which the cells 90 included in the cell image 10 are classified whether a normal cell or an abnormal cell by analyzing the cell image 10 by using the cell image analysis apparatus 100 (see FIG. 1) in this embodiment. In this embodiment, the cell image analysis apparatus 100 analyzes the cell image 10 by using the learned model 6 (see FIG. 1) to determine whether each cell 90 included in the cell image 10 is a normal cell or an abnormal cell. The learned model 6 is configured to output an area estimation image 12 when receiving the cell image 10. The area estimation image 12 is an image that shows a normal cell area 20 (see FIG. 7) and an abnormal cell area 21 (see FIG. 7) discriminatively from each other.

FIG. 4 is a block diagram showing a flow of image processing in this embodiment. As shown in FIG. 4, in this embodiment, the cell image analysis method roughly includes an image analysis method 101, a production method 102 of a first learned model 6a, and a production method 103 of a second learned model 6b.

### (Generation of Learning Model)

In the production method 102 of the first learned model 6a in this embodiment, the learned model 6 (first learned model 6a) is produced by training a first learning model 7a by using the corrected cell images 11. Specifically, in the production method 102 of the first learned model 6a, the first learned model 6a is produced by training a first learning model 7a by using teacher corrected cell images 30 and teacher area estimation images 31. In other words, in the production method 102 of the first learned model 6a, the teacher corrected cell images 30 as the corrected cell images 11 are provided as input data, and images as the teacher area estimation images 31 that include different label values attached to the normal cell area 20, the abnormal cell area 21 and the background area are provided as output data. As a result, in the production method 102 of the first learned model 6a, the first learning model 7a learns to classify whether each pixel of the input image into one of a normal cell, an abnormal cell, and a background. Specifically, the production method 102 of the first learned model 6a includes a step 102a of inputting teacher corrected cell images 30 into the first learning model 7a, and a step 102b of learning the first learning model 7a to output the teacher area estimation images 31 corresponding to the teacher corrected cell images 30. The first learned model 6a is a convolutional neural network (CNN) shown in FIG. 4 or a learning model partially including a convolutional neural network. The first learned model 6a produced by learning the first learning model 7a is stored in the storage 3 (FIG. 1) of the cell image analysis apparatus 100. The teacher corrected cell image 30 is the cell image 10 that is generated to reduce unevenness of brightness of the background 91.

In the production method 103 of the second learned model 6b, the second learned model 6b is produced by training the second learning model 7b by using teacher cell images 32 and teacher area estimation images 33. In this embodiment, the second learned model 6b analyzes whether each cell 90 is suitable for analysis whether the cell is a normal cell or an abnormal cell in non-corrected cell images 15, which are the cell images 10 to which correction is not applied. The non-corrected cell image 15 is an example of a “cell image to which correction is not applied” in the claims.

In other words, in the production method 103 of the second learned model 6b, the teacher cell images 32 as the non-corrected cell images 15 are provided as input data, and images as the teacher area estimation images 33 that include different label values attached to the normal cell area 20 (see FIG. 7), the abnormal cell area 21 (see FIG. 7) and the background area are provided as output data. As a result, in the production method 103 of the second learned model 6b, the second learning model 7b learns to classify whether each pixel of the input image into one of a normal cell, an abnormal cell, and a background. Specifically, the production method 102 of the second learn model 6b includes a step 103a of outputting teacher cell images 32 into the second learning model 7b, and a step 103b of learning the second learning model 7b to output the teacher area estimation images 33 corresponding to the teacher cell images 32. The second learned model 6b is a convolutional neural network shown in FIG. 4 or a learning model partially including a convolutional neural network. The second learned model 6b produced by learning the second learning model 7b is stored in the storage 3 (FIG. 1) of the cell image analysis apparatus 100. The teacher cell image 32 is the cell image 10 to which reduction processing for reducing unevenness of brightness irrespective of whether brightness of the background 91 is even or uneven.

### (Image Analysis Method)

In this embodiment, the image analysis method 101 classifies the cells 90 included in the cell image 10 acquired by the image acquirer 1 from the microscope 7 (see FIG. 1), etc., whether each cell is a normal cell or an abnormal cell. The image analysis method 101 according to this embodiment includes a step of acquiring the cell image 10 including the cells 90 (see FIG. 2); a step of generating the background component image 14 (see FIG. 5) that extracts a distribution of a brightness component of the background 91 (see FIG. 2) from the cell image 10; a step of generating a corrected cell image 11 (see FIG. 6) that is acquired by correcting the cell image 10 to reduce unevenness of brightness; and a first estimation step of estimating whether each cell 90 in the image is a normal cell or an abnormal cell by using the corrected cell image 11 and the learned model 6 (first learned model 6a) that has learned to analyze the cells 90. The steps in the image analysis method 101 will be described in detail later.

In this embodiment, the step of acquiring the cell image 10 is executed by the image acquirer 1. The image acquirer 1 is configured to acquire the cell images 10 from the imaging device such as the microscope 7 (see FIG. 1). Also, the image acquirer 1 is configured to output the acquired cell image 10 to the image analyzer 2b. Also, the image acquirer 1 is configured to output the acquired cell image 10 to the image processor 2c.

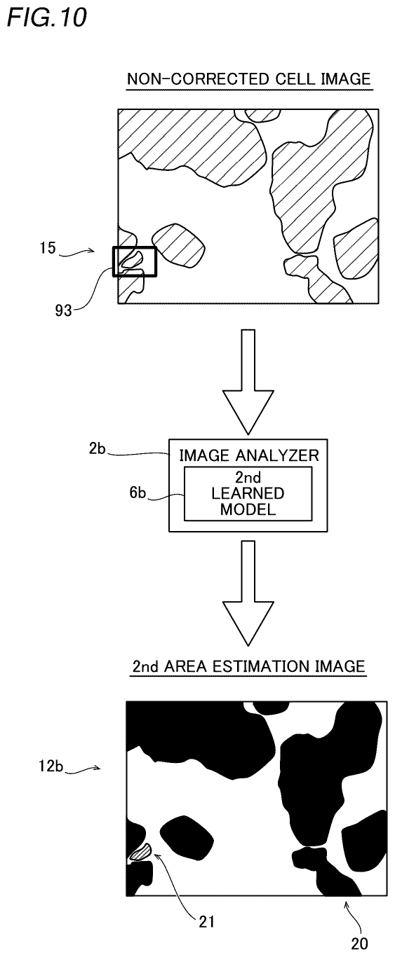

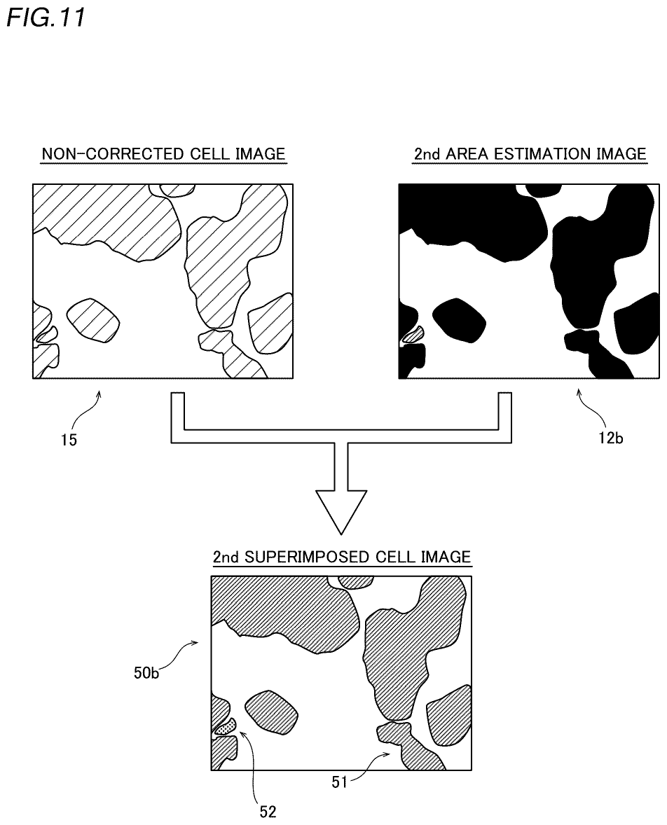

In this embodiment, the step of analyzing the non-corrected cell image 15 is executed by the image analyzer 2b as shown in FIG. 4. The image analyzer 2b is configured to output the corrected cell image 11 or the non-corrected cell image 15 to the learned model 6 to acquire the area estimation image 12. Specifically, the image analyzer 2b inputs the corrected cell image 11 to the first learned model 6a to acquire a first area estimation image 12a (see FIG. 7). Specifically, the image analyzer 2b inputs the non-corrected cell image 15 to the second learned model 6b to acquire a second area estimation image 12b (see FIG. 10). The controller 2a is configured to determine whether the first learned model 6a or the second learned model 6b is used for analysis by the image analyzer 2b. The image analyzer 2b is configured to output the acquired area estimation image 12 (first area estimation image 12a or second area estimation image 12b) to the superimposed cell image generator 2d.

The controller 2a is configured to determine whether the first learned model 6a or the second learned model 6b is used for analysis in accordance with an operating input from a user. Specifically, the controller 2a is configured to determine whether the first learned model 6a or the second learned model 6b is used for analysis in accordance with an operating input that selects whether to generate the corrected cell image 11 or not. That is, the controller 2a determines to use the first learned model 6a for the corrected cell images 11 and not to use the first learned model 6a for the non-corrected cell images 15. Also, the controller 2a determines to use the second learned model 6b for the non-corrected cell images 15 and not to use the second learned model 6b for the corrected cell images 11.

The superimposed cell image generator 2d is configured to generate the superimposed cell image 50 based on the cell image 10 or the corrected cell image 11 and the area estimation image 12. Also, the superimposed cell image generator 2d is configured to display the generated superimposed cell image 50 on the display 4.

### (Background Component Image)

A configuration of the image processor 2c that generates the background component image 14 is now described with reference to FIG. 5. In this embodiment, the image processor 2c generates the background component image 14 by extracting a distribution of brightness component of the background 91 from the cell image 10 by filtering the cell image 10 acquired by the image acquirer 1. Specifically, the image processor 2c generates the background component image 14 by applying a median filter to the cell image 10. In this embodiment, the image processor 2c applying a median filter to the cell image 10 for smoothing the cell image. In this embodiment, the image processor 2c extracts the background component by smoothing the image to make the cells 90 in the cell image 10 substantially unrecognizable.

In the filtering using the median filter, a median value of pixel values of pixels in an area (kernel) of a predetermined size centering a target pixel is acquired. Specifically, in the filtering using the median filter, pixel values of pixels in a kernel are acquired, and the acquired pixel values are sorter in increasing order so that a median value is acquired from the sorted pixel values. Then, the acquired median value is used as a pixel value of the target pixel. This filtering is applied to all the pixels of the cell image 10 by changing the target pixel one by one. For this reason, the filtering using the median filter increases a load of processing.

To address th, in this embodiment, the image processor 2c (see FIG. 1) generates a reduced cell image 10c by reducing the cell image 10a. Specifically, the image processor 2c generates the cell image 10c by applying lossless compression to the cell image 10a. In this embodiment, the image processor 2c reduces a size of the cell image 10a by ⅛, for example, to generate the reduced cell image 10c.

Subsequently, the image processor 2c generates a reduced background component image 13 by filtering the reduced cell image 10c. The reduced background component image 13 is an image that is generated by extracting unevenness of brightness (background component) of the background 91 by filtering. In the reduced cell image 10c, the cells 90 are shown by dashed lines representing that the cells 90 are unrecognizable.

In this embodiment, a kernel size of the median filter is set to a size that can extract the brightness component of the background 91 and can make the cell 90 in the cell image 10a unrecognizable after the smoothing. In other words, the kernel size of the median filter is set depending on a size of each cell 90 included in the cell image 10a. In this embodiment, the image processor 2c applies a median filter to the cell image 10c that is acquired by reducing the cell image 10a. For this reason, the kernel size of the median filter is set depending on a magnification of the cell image 10c. Accordingly, because the filtering can be applied by using a kernel size suitably adjusted for the magnification of the cell image 10c, it is possible to prevent increase of the load of processing caused by increase of the number of processes for extracting pixel values and sorting the pixel values due to a too large kernel size. Also, it is possible to prevent inaccurate extraction of the background component caused by a too small small kernel size.

Subsequently, the image processor 2c generates the background component image 14 by enlarging the reduced background component image 13. Specifically, the image processor 2c enlarges the reduced background component image 13 so that a sized of the background component image 14 is increased to the same size as the cell image 10a. In this embodiment, the image processor 2c generates the background component image 14 by enlarging the reduced background component image 13 by 8 times, for example. In the background component image 14, the component of the background 91 is extracted by filtering using the median filter so that the cells 90 are unrecognizable. Also, in the background component image 14 shown in FIG. 5, the cells 90 are indicated by dashed lines representing that the cells 90 are unrecognizable.

### (Corrected Cell Image)

A configuration of the image processor 2c (see FIG. 1) that generates the corrected cell image 11 is now described with reference to FIG. 6. In this embodiment, the image processor 2c generates the corrected cell image 11 by correcting the cell image 10 to reduce unevenness of brightness based on the cell image 10a and the background component image 14. Specifically, as shown in FIG. 6, the image processor 2c subtracts the background component image 14 from the cell image 10a. When the background component image 14 is subtracted from the cell image 10a, pixel values of the entire image are reduced so that contrast of the image may be reduced. To address this, in this embodiment, the image processor 2c subtracts the background component image 14 from the cell image 10a, and then adds a predetermined brightness value to generate the corrected cell image 11. The predetermined pixel value is, for example, a half the gray-scale value of the pixel value of the cell image 10a. In this embodiment, for example, if the pixel value of the cell image 10a is represented by a value of 256 levels of gray, the image processor 2c adds 128 as the predetermined pixel value.

As shown in FIG. 6, unevenness of brightness of the background 91 is reduced in the corrected cell image 11. The background 91 without hatching pattern in the corrected cell image 11 shown in FIG. 6 represents the reduction of unevenness of brightness of the background 91.

### (First Area Estimation Image)

A configuration of the image analyzer 2b that acquires the first area estimation image 12a is now described with reference to FIG. 7. In this embodiment, the image analyzer 2b acquires the first area estimation image 12a by inputting the corrected cell image 11 to the first learned model 6a. Specifically, the image analyzer 2b estimates the normal cell area 20, which is an area of a normal cell, and the abnormal cell area 21, which is an area of an abnormal cell, based on an estimation result of the first learned model 6a. In this embodiment, the image analyzer 2b is configured to generate, as the first area estimation image 12a, an image that shows the normal cell area 20, the abnormal cell area 21 and the background area discriminatively from each other. Specifically, the image analyzer 2b displays the normal cell area 20, the abnormal cell area 21 and the background area with different colors applied thereon in the first area estimation image 12a so that the normal cell area 20, the abnormal cell area 21 and the background area can be discriminated from each other. In an exemplary case shown in FIG. 7, the image analyzer 2b displays the normal cell area 20 in black, the abnormal cell area 21 in gray, and the background area in white to generate the first area estimation image 12a, which shows the normal cell area 20, the abnormal cell area 21 and the background area discriminatively from each other. In the first area estimation image 12a, the abnormal cell area 21 is shown by hatching that represents gray representing the abnormal cell area 21. In the exemplary case shown in FIG. 7, a user can recognize that the cell 90 that located in an area 92 in the corrected cell image 11 is abnormal cells when seeing the first area estimation image 12a.

### (First Superimposed Cell Image)

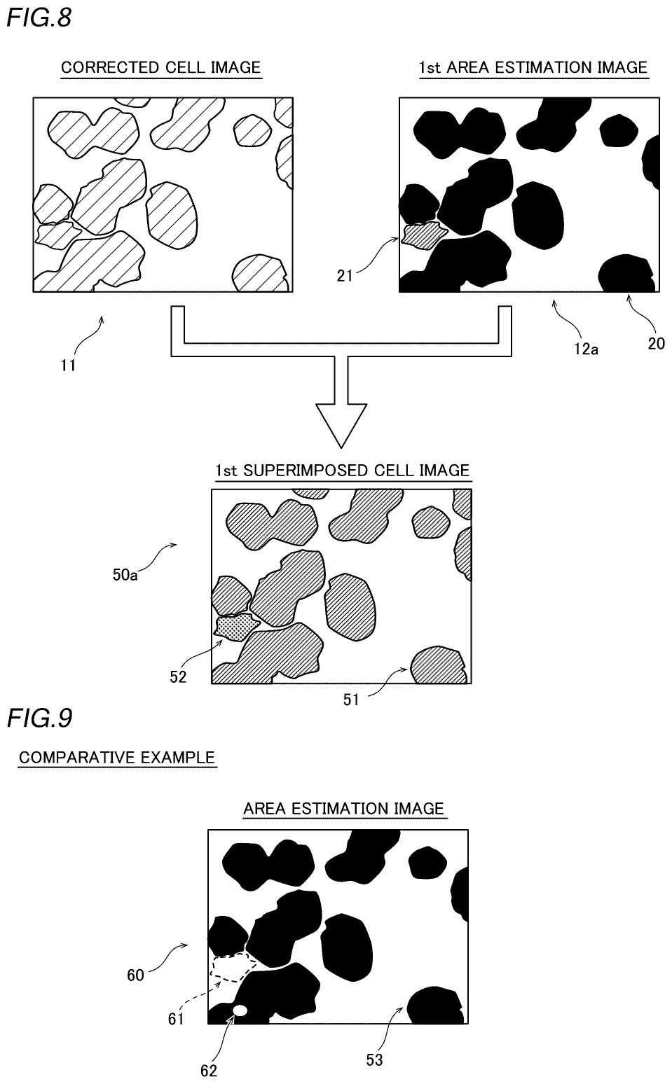

A configuration of the superimposed cell image generator 2d (see FIG. 1) that generates the superimposed cell image 50 is now described with reference to FIG. 8. In this embodiment, the image processor 2c is configured to generate the superimposed cell image 50 by superimposing a first mark 51 indicating the normal cell area 20 (see FIG. 7) and a second mark 52 indicating the abnormal cell area 21 (see FIG. 7) on the corrected cell image 11. Specifically, the superimposed cell image generator 2d is configured to generate the first superimposed cell image 50a based on the corrected cell image 11 and the first area estimation image 12a. The superimposed cell image generator 2d acquires the first mark 51 based on the normal cell area 20 in the first area estimation image 12a. The superimposed cell image generator 2d acquires the second mark 52 based on the abnormal cell area 21 in the first area estimation image 12a. Subsequently, the superimposed cell image generator 2d generates the first superimposed cell image 50a by superimposing the first mark 51 and the second mark 52, which are acquired based on the first area estimation image 12a, on the corrected cell image 11.

Here, it is conceivable that a normal cell and an abnormal cell are stained with different colors to discriminate whether the cell 90 is the normal cell and the abnormal cell. However, in a case in which a normal cell and an abnormal cell are stained with different colors to discriminate between the normal cell and the abnormal cell, cells 90 are destroyed. If the cells 90 are destroyed, the cell 90 cannot be used for passaging, etc. To address this, as shown in FIG. 8, the superimposed cell image generator 2d generates the first superimposed cell image 50a that can discriminate between normal and abnormal cell areas 20 and 21 by showing the first mark 51 and the second mark 52 discriminatively from each other. The superimposed cell image generator 2d displays the normal cell area 20 and the abnormal cell area 21 discriminatively from each other by showing the normal cell area 20 and the abnormal cell area 21 in different colors. The superimposed cell image generator 2d shows the first mark 51 in blue and the second mark 52 in red, for example. In an exemplary case shown in FIG. 8, different hatching patterns applied to the first mark 51 and the second mark 52 represent different colors applied to the normal and abnormal cell areas 20 and 21 in the first superimposed cell image 50a.

### (Area Estimation Image in Comparative Example)

An area estimation image 60 in a comparative example is now described with reference to FIG. 9. An area estimation image 60 in the comparative example is an image that shows a normal cell area 20 (see FIG. 7) and an abnormal cell area 21 (see FIG. 7) discriminatively from each other based on analysis of the cell image 10 including uneven brightness of the background 91 (see FIG. 3) analyzed by the learned model. The area estimation image 60 in the comparative example is an image that shows a normal cell area 20 and an abnormal cell area 21 estimated by the learned model without correcting the cell image 10a (see FIG. 3) used to generate the first area estimation image 12a (see FIG. 8). Accuracy of estimation in the comparative area estimation image 60 is reduced by the unevenness of brightness of the background 91. Specifically, accuracy of estimation in a left-side area of the image is reduced by the unevenness of brightness of the background 91.

Specifically, as comparison between the first area estimation image 12a shown in FIG. 8 and the area estimation image 60 shown in FIG. 9, an area 61 shown by a dashed line in the area estimation image 60 is not estimated as the abnormal cell area 21 (see FIG. 7). Also, an area 62 shown by a solid line in the normal cell area 20 (see FIG. 7) is not estimated as the normal cell area 20. That is, the area estimation image 60 in the comparative example includes the area 61, which does not accurately detect the abnormal cell area 21, and the area 62, which does not accurately detect the normal cell area 20. Consequently, it can be seen that accuracy of analysis by the learned model in the comparative example is reduced. Contrary to this, reduction of accuracy of the classification into the normal cell area 20 or the abnormal cell area 21 can be prevented in the first area estimation image 12a in this embodiment.

### (Second Area Estimation Image)

A configuration of the image analyzer 2b that acquires the second area estimation image 12b is now described with reference to FIG. 10. In this embodiment, the image analyzer 2b acquires the second area estimation image 12b by inputting the non-corrected cell image 15 to which correction is not applied to the second learned model 6b. Specifically, the image analyzer 2b estimates the normal cell area 20, which is an area of a normal cell, and the abnormal cell area 21, which is an area of an abnormal cell, based on an estimation result of the second learned model 6b. The image analyzer 2b is configured to generate, as the second area estimation image 12b, an image that shows the normal cell area 20, the abnormal cell area 21 and the background area discriminatively from each other.

Specifically, the image analyzer 2b displays the normal cell area 20, the abnormal cell area 21 and the background area with different colors applied thereon in the second area estimation image 12b so that the normal cell area 20, the abnormal cell area 21 and the background area can be discriminated from each other. In an exemplary case shown in FIG. 10, the image analyzer 2b displays the normal cell area 20 in black, the abnormal cell area 21 in gray, and the background area in white to generate the second area estimation image 12b, which shows the normal cell area 20, the abnormal cell area 21 and the background area discriminatively from each other. In the second area estimation image 12b, the abnormal cell area 21 is shown by hatching that represents gray representing the abnormal cell area 21. In the exemplary case shown in FIG. 10, a user can recognize that the cell 90 that located in an area 93 in the non-corrected cell image 15 is abnormal cells when seeing the second area estimation image 12b.

### (Second Superimposed Cell Image)

A configuration of the superimposed cell image generator 2d (see FIG. 1) that generates the second superimposed cell image 50b is now described with reference to FIG. 11. In this embodiment, the superimposed cell image generator 2d is configured to generate the second superimposed cell image 50b by superimposing a first mark 51 indicating the normal cell area 20 (see FIG. 10) and a second mark 52 indicating the abnormal cell area 21 (see FIG. 10) on the non-corrected cell image 15. Specifically, the superimposed cell image generator 2d acquires the first mark 51 based on the normal cell area 20 in the second area estimation image 12b. The superimposed cell image generator 2d acquires the second mark 52 based on the abnormal cell area 21 in the second area estimation image 12b. Subsequently, the superimposed cell image generator 2d generates the second superimposed cell image 50b by superimposing the first mark 51 and the second mark 52, which are acquired based on the second area estimation image 12b, on the non-corrected cell image 15.

As shown in FIG. 11, the superimposed cell image generator 2d generates the second superimposed cell image 50b that can discriminate between the normal cell area 20 and the abnormal cell area 21 by showing the first mark 51 and the second mark 52 discriminatively from each other. The superimposed cell image generator 2d displays the normal cell area 20 and the abnormal cell area 21 discriminatively from each other by showing the normal cell area 20 and the abnormal cell area 21 in different colors. The superimposed cell image generator 2d shows the first mark 51 in blue and the second mark 52 in red, for example. In an exemplary case shown in FIG. 11, different hatching patterns applied to the first mark 51 and the second mark 52 represent different colors applied to the normal cell area 20 and the abnormal cell area 21 in the second superimposed cell image 50b.

### (Display of Superimposed Cell Image)

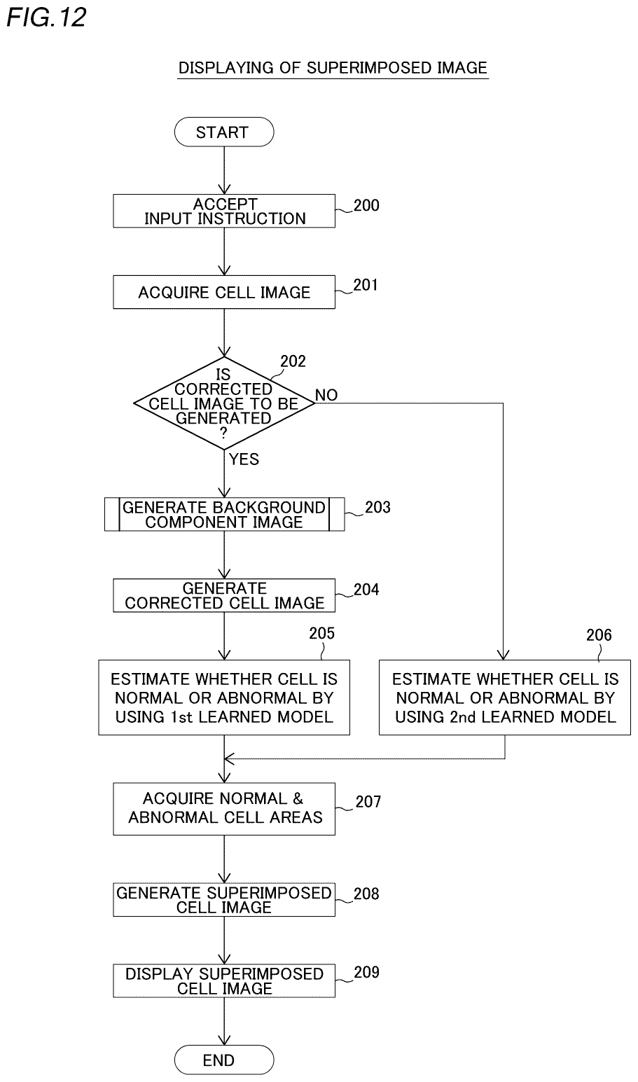

Processes of displaying the superimposed cell image 50 in the cell image analysis apparatus 100 is now described with reference to FIG. 12.

In step 200, the controller 2a accepts a user input instruction to select whether to generate the corrected cell image 11 or not. In this embodiment, the controller 2a accepts an input instruction from a user through the input acceptor 5 (see FIG. 1) to select whether to generate the corrected cell image 11 or not. The controller 2a sets selection whether to generate the corrected cell image 11 or not in accordance with the input instruction from the user to select whether to generate the corrected cell image 11 or not. Also, the controller 2a stores the setting of selection whether to generate the corrected cell image 11 or not in the storage 3.

In step 201, the image acquirer 1 acquires a cell image 10 including cells 90.

In step 202, the controller 2a determines whether to generate the corrected cell image 11 or not in accordance with the input instruction accepted in step 200. If the corrected cell image 11 is to be generated, the procedure goes to step 203. If the corrected cell image 11 is not to be generated, the procedure goes to step 206. In this embodiment, the controller 2a determines whether to generate the corrected cell image 11 or not by checking the setting of selection whether to generate the corrected cell image 11 or not stored in the storage 3.

In step 203, the image processor 2c generates the background component image 14 by extracting a distribution of brightness component of the background 91 from the cell image 10 by filtering the acquired cell image 10.

In step 204, the image processor 2c generates the corrected cell image 11 by correcting the cell image 10 to reduce unevenness of brightness based on the cell image 10 and the background component image 14. In this embodiment, as shown in FIG. 6, in step 204, the image processor 2c subtracts the background component image 14 from the cell image 10, and then adds a predetermined brightness value to generate the corrected cell image 11.

In step 205, the image analyzer 2b executes a first estimation step of estimating whether each cell 90 in the image is a normal cell or an abnormal cell by using the corrected cell image 11 and the learned model 6 that has learned to analyze the cells 90. In this embodiment, if the corrected cell image 11 is to be generated, the image analyzer 2b applies the first estimation step to the corrected cell image 11 by using a first learned model 6a that is produced by using the corrected cell image 11 (teacher corrected cell image 30) as the learned model 6.

If the procedure goes from step 202 to step 206, the image analyzer 2b applies processes of the second estimation step to the cell image 10 in step 206 by using the second learned model 6b produced by using the non-corrected cell images 15 (teacher cell images 32).

In step 207, the image processor 2c estimates the normal cell area 20, which is an area of a normal cell, and the abnormal cell area 21, which is an area of an abnormal cell, based on an estimation result of the learned model 6. Specifically, in step 207, the image processor 2c acquires an area estimation image 12 showing the normal cell area 20 and the abnormal cell area 21 discriminatively from each other.

In step 208, the superimposed cell image generator 2d is configured to generate the superimposed cell image 50 by superimposing a first mark 51 indicating the normal cell area 20 and a second mark 52 indicating the abnormal cell area 21 on the corrected cell image 11.

In step 209, the controller 2a directs the display 4 to display a superimposed cell image 50. In other words, the controller 2a displays the superimposed cell image 50 on the display 4 to show the normal cell area 20 and the abnormal cell area 21 discriminatively from each other.

In this embodiment, if the corrected cell image 11 is to be generated, the cell image analysis apparatus 100 executes step 204 of generating the corrected cell image 11, and then executes processes of the first estimation step (processing of step 205) by using the corrected cell image 11 and the learned model 6. If the corrected cell image 11 is not to be generated, the cell image analysis apparatus 100 executes processes of the second estimation step (processing of step 206) for estimation by using the cell image 10 and the learned model 6 without executing step 203 of generating the background component image 14.

### (Generation of Background Component Image)

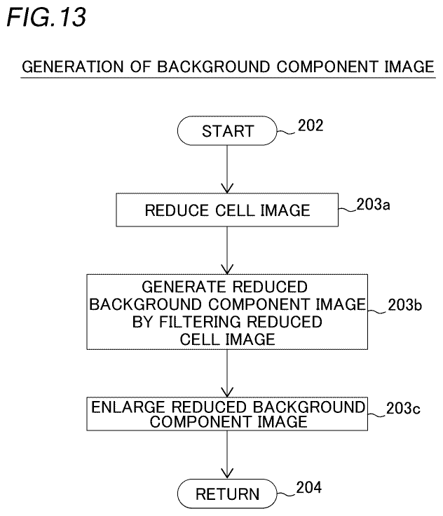

Processing of the image processor 2c that generates the background component image 14 is now described with reference to FIG. 13.

In step 203a, the image processor 2c reduces the cell image 10a. Specifically, the image processor 2c generates the reduced cell image 10c by applying lossless compression to the cell image 10a.

In step 203b, the image processor 2c generates the reduced background component image 13 by filtering the reduced cell image 10c. In this embodiment, the image processor 2c generates the reduced background component image 13 by filtering the reduced cell image 10c by using the median filter.

In step 203c, the image processor 2c enlarges the reduced background component image 13. That is, the image processor 2c generates the background component image 14 by enlarging the reduced background component image 13. Subsequently, the procedure goes to step 204.

### Advantages of the Embodiment

In this embodiment, the following advantages are obtained.

In this embodiment, as discussed above, the cell image analysis method includes a step of acquiring a cell image 10 including cells 90; a step of generating a background component image 14 that extracts a distribution of a brightness component of a background 91 from the cell image 10 by filtering the acquired cell image 10; a step of generating a corrected cell image 11 that is acquired by correcting the cell image 10 based on the cell image 10 and the background component image 14 to reduce unevenness of brightness; and a first estimation step of estimating whether the cell 90 in the image is a normal cell or an abnormal cell by using the corrected cell image 11 and a learned model 6 that has learned to analyze the cell 90.

Accordingly, this cell image analysis method can estimate whether a cell 90 in an image is a normal cell or an abnormal cell by using the corrected cell image 11 generated to reduce unevenness of brightness of the background 91 and the learned model 6. Consequently, it is possible to prevent reduction of accuracy of analysis by the learned model 6 caused by unevenness of brightness of the background 91 of the cell image 10.

In addition, following additional advantages can be obtained by the aforementioned embodiment added with configurations discussed below.

That is, in this embodiment, as discussed above, the cell image 10 is an image including cells 90 that are cultivated in cultivation solution 81 with which the cultivation container 80 is filled, and are located in a near-edge area 70 of the cultivation container 80. A height of a liquid surface 81a of the cultivation solution 81 varies in a near-edge area 70 of the cultivation container 80, specifically, the height of the liquid surface 81a of the cultivation solution 81 is increased toward the near-edge area 70 of the cultivation container 80 by surface tension. In a case in which the height of the liquid surface 81a of the cultivation solution 81 varies, height difference of the cultivation solution 81 causes uneven brightness of the background 91 shown in the cell image 10. To address this, even in a case of analysis of the cell images 10 that includes unevenness of brightness of the background 91 caused by variation of the height of the liquid surface 81a of the cultivation solution 81 in the near-edge area 70 of the cultivation container 80, reduction of accuracy of analysis by the learned model 6 can be easily prevented by applying the present invention to the cell image 10 including the cultivated cells 90 that are located in the near-edge area 70 of the cultivation container 80,

In this embodiment, as described above, the filtering is a process of applying a median filter to the cell image 10 to generate the background component image 14. Here, in a case in which a Gaussian filter or an averaging filter is applied as the filtering, for example, stray light that causes extremely high pixel values or foreign objects that cause extremely low pixel values may prevent accurate acquisition of a brightness component of the background 91. To address this, a median filter, which uses a median value of pixel values for each pixel in a predetermined area for smoothing, is applied so that the brightness component of the background 91 can be accurately acquired even in a case in which the background includes such stray light or foreign objects. Consequently, it is possible to acquire the corrected cell image 11, which accurately corrects the unevenness of brightness of the background 91.

In this embodiment, as discussed above, a step of reducing the cell image 10 is further provided; in the step of generating a background component image 14, a reduced background component image 13 is generated by filtering the reduced cell image 10c; and a step of increasing the reduced background component image 13 is further provided. Accordingly, because the reduced cell image 10c is filtered, it is possible to prevent increase of a load of the filtering. In a case in which a median filter, which uses a median value of pixel values for each pixel in a predetermined area for smoothing, is applied, it is possible to further prevent increase of a load of the filtering.

In this embodiment, as discussed above, a step of accepting a user input instruction to select whether to generate the corrected cell image 11 or not is further provided; if the corrected cell image 11 is to be generated, the step of generating a corrected cell image 11 is executed, and the first estimation step is executed by using the corrected cell image 11 and the learned model 6 (first learned model 6a); and a second estimation step of estimating whether the cell in the image is a normal cell or an abnormal cell by using the cell image 10 (non-corrected cell image 15) and the learned model 6 (second learned model 6b) without executing the step of generating a background component image 14 if the corrected cell image 11 is not to be generated is further provided. Accordingly, because the first estimation step is executed by using the corrected cell image 11 and the first learned model 6a if the corrected cell image 11 is to be generated, it is possible to prevent reduction of accuracy of analysis by the learned model 6 (first learned model 6a) caused by unevenness of brightness of the background 91 of the cell image 10. Also, because the second estimation step is executed by using the non-corrected cell image 15 and the second learned model 6b if the corrected cell image 11 is not to be generated, the non-corrected cell image 15 can be analyzed without generating the background component image 14. Because background component image 14 is not generated, it is possible to correspondingly prevent increase of a load of filtering.

In this embodiment, as discussed above, if the corrected cell image 11 is to be generated, the first estimation step is applied to corrected cell image 11 by using a first learned model 6a that has been produced by using the corrected cell images 11 as the learned model 6; and if the corrected cell image 11 is not to be generated, the second estimation step is applied to the cell image 10 by using a second learned model 6b that is produced by using the cell images 10 (non-corrected cell image 15) to which correction is not applied. Accordingly, because the first estimation step is executed if the corrected cell image 11 is analyzed, it is possible to analyze the corrected cell by using the first learned model 6a, which is suitable for analyzing the corrected cell image 11. Consequently, it is possible to prevent reduction of accuracy of analysis n a case in which the cell image 10 that has uneven brightness of the background 91 is analyzed. Here, if the cell image 10 is filtered to reduce the unevenness of brightness, outlines of the cells 90 become unclear so that contrast of the cells 90 decreases. To address this, because the second estimation step is executed by using the non-corrected cell image 15 and the second learned model 6b if the cell images 10 (non-corrected cell image 15) to which correction is not applied is analyzed, the non-corrected cell image 15, which is not filtered, can be analyzed by the second learned model 6b. As a result, because the non-corrected cell image 15 is not filtered, the non-corrected cell images 15 can be analyzed while preventing reduction of contrast of the cells 90.

In this embodiment, as discussed above, in the step of generating a corrected cell image 11, the corrected cell image 11 is generated by subtracting the background component image 14 from the cell image 10 and by adding a predetermined brightness value. Accordingly, because a predetermined brightness value is added dissimilar to a configuration in which the background component image 14 is simply subtracted from the cell image 10, it is possible to prevent reduction of contrast of the corrected cell image 11.

In this embodiment, as discussed above, in the step of estimating whether the cell 90 included in the corrected cell image 11 is a normal cell or an abnormal cell, a normal cell area 20, which is an area of the normal cell, and an abnormal cell area 21, which is an area of the abnormal cell, are estimated based on an estimation result estimated by the learned model 6; and a step of displaying the normal cell area 20 and the abnormal cell area 21 discriminatively from each other is further provided. Because the normal cell area 20 and the abnormal cell area 21 are displayed discriminatively from each other in the cell image 10 that includes reduced unevenness of brightness of the background 91, users can easily discriminate between the normal cell area 20 and the abnormal cell area 21.

In this embodiment, as discussed above, a step of generating a superimposed cell image 50 by superimposing a first mark 51 indicating the normal cell area 20 and a second mark 52 indicating the abnormal cell area 21 on the corrected cell image 11 is further provided. The first mark 51 and the second mark 52 shown in the superimposed cell image 50 allow users to easily discriminate between the normal cell area 20 and the abnormal cell area 21.

In this embodiment, as discussed above, a step of producing a learned model 6 (first learned model 6a) by training a first learning model 7a by using the corrected cell images 11. Accordingly, it is possible to generate the learned model 6 (first learned model 6a) suitable for analyzing the corrected cell image 11 that includes reduced unevenness of brightness of the background 91. Consequently, it is possible to prevent reduction of accuracy of analysis of the corrected cell image 11.

### MODIFIED EMBODIMENTS

Note that the embodiment disclosed this time must be considered as illustrative in all points and not restrictive. The scope of the present invention is not shown by the above description of the embodiments but by the scope of claims for patent, and all modifications (modified embodiments) within the meaning and scope equivalent to the scope of claims for patent are further included.

While the example in which the image processor 2c generates the background component image 14 by applying a median filter to the cell image 10 has been shown in the aforementioned embodiment, the present invention is not limited to this. For example, image processor 2c may be configured to generate the background component image 14 by applying a Gaussian filter or an averaging filter to the cell image 10. However, in a case in which the image processor 2c generates the background component image 14 by applying a Gaussian filter or an averaging filter, stray light that causes extremely high pixel values or foreign objects that cause extremely low pixel values may prevent accurate acquisition of a brightness component of the background 91. For this reason, the image processor 2c is preferably configured to generate the background component image 14 by applying a median filter to the cell image.

While the example in which the image processor 2c generates the background component image 14 by reducing the cell image 10a and by filtering the reduced cell image 10c has been shown in the aforementioned embodiment, the present invention is not limited to this. The image processor 2c may be configured to generate the background component image 14 by filtering the cell image 10a without reducing the cell image 10a. However, if the image processor 2c is configured to generate the background component image 14 without reducing the cell image 10a, a load of filtering increases. For this reason, the image processor 2c is preferably configured to generate the background component image 14 by filtering the reduced cell image 10c.

While the example in which the controller 2a determines whether to generate the corrected cell image 11 or not in accordance an input instruction to select whether to generate the corrected cell image 11 or not has been shown in the aforementioned embodiment, the present invention is not limited to this. For example, the controller 2a may be configured to determine whether brightness of the background 91 of the cell image 10 is uneven, and then to determine to generate the corrected cell image 11 if brightness of the background 91 of the cell image 10 is uneven and to determine not to generate the corrected cell image 11 if brightness of the background 91 of the cell image 10 is not uneven.

While the example in which the image processor 2c switches between activation and deactivation of generation of the corrected cell image 11 in accordance with the determination by the controller 2a has been shown in the aforementioned embodiment, the present invention is not limited to this. The image processor 2c may be configured to generate the corrected cell image 11 not based on the determination by the controller 2a but on all occasions. However, if the image processor 2c is configured to generate the corrected cell images 11 on all occasions, the cell images 10b that do not include unevenness of brightness of the background 91 are filtered. Accordingly, a load of processing increases. Consequently, the image processor 2c preferably switches between activation and deactivation of generation of the corrected cell image 11 in accordance with the determination by the controller 2a

While the example in which the image processor 2c generates the corrected cell image 11 by correcting the cell image 10a that includes unevenness of brightness of the background 91, and does not correct the cell image 10b that does not include unevenness of brightness of the background 91 has been shown in the aforementioned embodiment, the present invention is not limited to this. The image processor 2c may be configured to correct the cell image 10 irrespective of whether the cell image includes unevenness of brightness of the background 91 or not. However, even if cell images 10 that do not include unevenness of brightness of the background 91 are corrected, contrast of cells 90 is reduced by smoothing the cells 90. For this reason, the image processor 2c is preferably configured to correct only the cell image 10 that includes unevenness of brightness of the background 91.

While the example in which the image processor 2c subtracts the background component image 14 from the cell image 10a to generate the corrected cell image 11 has been shown in the aforementioned embodiment, the present invention is not limited to this. For example, the image processor 2c may be configured to divide the cell image 10a by the background component image 14 to generate the corrected cell image 11. The image processor 2c can use any technique to generate the corrected cell image 11.

While the example in which the image processor 2c subtracts the background component image 14 from the cell image 10a, and then adds a predetermined brightness value to generate the corrected cell image 11 has been shown in the aforementioned embodiment, the present invention is not limited to this. For example, the image processor 2c may be configured to subtract the background component image 14 from the cell image 10a to generate the corrected cell image 11 without adding the predetermined brightness value. However, if the image processor 2c subtracts the background component image 14 from the cell image 10a to generate the corrected cell image 11 without adding the predetermined brightness value, contrast of the corrected cell image 11 decreases. For this reason, the image processor 2c is preferably configured to subtract the background component image 14 from the cell image 10a, and then adds a predetermined brightness value to generate the corrected cell image 11.

While the example in which the superimposed cell image generator 2d the superimposed cell image 50 that shows the normal cell area 20 and the abnormal cell area 21 discriminatively from each other by showing the normal cell area 20 in blue and the abnormal cell area 21 in red has been shown in the aforementioned embodiment, the present invention is not limited to this. As long as the normal cell area 20 and the abnormal cell area 21 can be discriminated, the superimposed cell image generator 2d can show the normal cell area 20 and the abnormal cell area 21 in any different colors.

While the example in which the cell image analysis apparatus 100 produces the first learned model 6a and the second learned model 6b has been shown in the aforementioned embodiment, the present invention is not limited to this. For example, the cell image analysis apparatus 100 may be configured to use the first learned model 6a and the second learned model 6b that are produced by a separated analysis apparatus other than the cell image analysis apparatus 100.

While the example in which the image analyzer 2b analyzes the corrected cell image 11 by using the first learned model 6a, and analyzes the non-corrected cell image 15 by using the second learned model 6b has been shown in the aforementioned embodiment, the present invention is not limited to this. For example, the image analyzer 2b may not execute analysis using the second learned model 6b. However, in a casein which the image analyzer 2b does not execute analysis using the second learned model 6b, the non-corrected cell image 15 is analyzed by the first learned model 6a. In this case, contrast of the cells 90 in the non-corrected cell image 15 may be reduced. For this reason, the image analyzer 2b is preferably configured to analyze the corrected cell image 11 by using the first learned model 6a, and to analyze the non-corrected cell image 15 by using the second learned model 6b

While the example in which the image acquirer 1 acquires the cell Image 10 in step 201 has been shown in the aforementioned embodiment, the present invention is not limited to this. For example, the image processor 2c may be configured to acquire the cell image 10 that is previously acquired by the image acquirer 1 and stored in the storage 3.

While the example in which the image analyzer 2b is configured to analyze whether each cell 90 included in the cell image 10 is an undifferentiated cell or a deviated cell has been shown in the aforementioned embodiment, the present invention is not limited to this. For example, the image analyzer 2b may be configured to analyze whether each cell is a cancer cell and a non-cancer cell. The cells analyzed by the image analyzer 2b are not limited to undifferentiated cells and undifferentiated deviated cells.

### [Modes]

The aforementioned exemplary embodiment will be understood as concrete examples of the following modes by those skilled in the art.

### (Mode Item 1)

A cell image analysis method includes a step of acquiring a cell image including a cell; a step of generating a background component image that extracts a distribution of a brightness component of a background from the cell image by filtering the acquired cell image; a step of generating a corrected cell image that is acquired by correcting the cell image based on the cell image and the background component image to reduce unevenness of brightness; and a first estimation step of estimating whether the cell in the image is a normal cell or an abnormal cell by using the corrected cell image and a learned model that has learned to analyze the cell.

### (Mode Item 2)

In the cell image analysis method according to mode item 1, the cell image is an image including a cell that is cultivated in a cultivation solution with which the cultivation container is filled, and is located in a near-edge area of the cultivation container as the cell.

### (Mode Item 3)

In the cell image analysis method according to mode item 1 or 2, the filtering is a process of applying a median filter to the cell image to generate the background component image.

### (Mode Item 4)

In the cell image analysis method according to any of mode items 1 to 3, a step of reducing the cell image is further provided; and in the step of generating a background component image, a reduced background component image is generated as the background component image by filtering the reduced cell image; and a step of increasing the reduced background component image is further provided.

### (Mode Item 5)

In the cell image analysis method according to any of mode items 1 to 4, a step of accepting a user input instruction to select whether to generate the corrected cell image or not is further provided; if the corrected cell image is to be generated, the step of generating a corrected cell image is executed, and the first estimation step is executed by using the corrected cell image and the learned model; and a second estimation step of estimating whether the cell in the image is a normal cell or an abnormal cell by using the cell image and the learned model without executing the step of generating a background component image if the corrected cell image is not to be generated is further provided.

### (Mode Item 6)

In the cell image analysis method according to mode item 5, if the corrected cell image is to be generated, the first estimation step is applied to the corrected cell image by using a first learned model that has been produced by using corrected cell images as the learned model; and if the corrected cell image is not to be generated, the second estimation step is applied to the cell image by using a second learned model that has been produced by using cell images to which correction is not applied.

### (Mode Item 7)

In the cell image analysis method according to any of mode items 1 to 6, in the step of generating a corrected cell image, the corrected cell image is generated by subtracting the background component image from the cell image and by adding a predetermined brightness value.

### (Mode Item 8)

In the cell image analysis method according to any of mode items 1 to 7, in the step of estimating whether the cell included in the corrected cell image is a normal cell or an abnormal cell, a normal cell area, which is an area of the normal cell, and an abnormal cell area, which is an area of the abnormal cell, are estimated based on an estimation result estimated by the learned model; and a step of displaying the normal cell area and the abnormal cell area discriminatively from each other is further provided.

### (Mode Item 9)

In the cell image analysis method according to mode item 8, a step of generating a superimposed cell image by superimposing a first mark indicating the normal cell area and a second mark indicating the abnormal cell area on the corrected cell image is further provided.

### (Mode Item 10)

In the cell image analysis method according to mode item 1, a step of producing the learned model by training a learning model by using corrected cell images is further provided.

### DESCRIPTION OF THE SIGN

6; learned model
6a; first learned model
6b; second learned model
10; cell image
10c; reduced cell image
13; reduced background component image
14; background component image
15; non-corrected cell image
20; normal cell area
21; abnormal cell area
50; superimposed image
51; first mark
52; second mark
70; near-edge area of cultivation container
80; cultivation container
80a; edge of cultivation container
81; cultivation solution
90; cell (cultivated cell)
91; background

1. A cell image analysis method comprising:
a step of acquiring a cell image including a cell;
a step of generating a background component image that extracts a distribution of a brightness component of a background from the cell image by filtering the acquired cell image;
a step of generating a corrected cell image that is acquired by correcting the cell image based on the cell image and the background component image to reduce unevenness of brightness; and
a first estimation step of estimating whether the cell in the image is a normal cell or an abnormal cell by using the corrected cell image and a learned model that has learned to analyze the cell.

2. The cell image analysis method according to claim 1, wherein the cell image is an image including a cell that is cultivated in a cultivation solution with which the cultivation container is filled, and is located in a near-edge area of the cultivation container as the cell.

3. The cell image analysis method according to claim 1, wherein the filtering is a process of applying a median filter to the cell image to generate the background component image.

4. The cell image analysis method according to claim 1 further comprising a step of reducing the cell image, wherein
in the step of generating a background component image, a reduced background component image is generated as the background component image by filtering the reduced cell image; and
the cell image analysis method further comprises a step of increasing the reduced background component image.

5. The cell image analysis method according to claim 1 further comprising a step of accepting a user input instruction to select whether to generate the corrected cell image or not, wherein
if the corrected cell image is to be generated, the step of generating a corrected cell image is executed, and the first estimation step is executed by using corrected cell images and the learned model; and
the cell image analysis method further comprises a second estimation step of estimating whether the cell in the image is a normal cell or an abnormal cell by using the cell image and the learned model without executing the step of generating a background component image if the corrected cell image is not to be generated.

6. The cell image analysis method according to claim 5, wherein
if the corrected cell image is to be generated, the first estimation step is applied to the corrected cell image by using a first learned model that has been produced by using corrected cell images as the learned model; and
if the corrected cell image is not to be generated, the second estimation step is applied to the cell image by using a second learned model that has been produced by using cell images to which correction is not applied.

7. The cell image analysis method according to claim 1, wherein in the step of generating a corrected cell image, the corrected cell image is generated by subtracting the background component image from the cell image and by adding a predetermined brightness value.

8. The cell image analysis method according to claim 1, wherein
in the step of estimating whether the cell included in the corrected cell image is a normal cell or an abnormal cell, a normal cell area, which is an area of the normal cell, and an abnormal cell area, which is an area of the abnormal cell, are estimated based on an estimation result estimated by the learned model; and
the cell image analysis method further comprises a step of displaying the normal cell area and the abnormal cell area discriminatively from each other.

9. The cell image analysis method according to claim 8 further comprising a step of generating a superimposed cell image by superimposing a first mark indicating the normal cell area and a second mark indicating the abnormal cell area on the corrected cell image.

10. The cell image analysis method according to claim 1 further comprising a step of producing the learned model by training a learning model by using corrected cell images.
